(19) United States
(12) Patent Application Publication
(10) Pub. No.: US 2005/0049231 A1
(43) Pub. Date: Mar. 3, 2005

(54) Treatment method

(76) Inventors: Peter Knox, Berkshire RG1 5TU (GB); Helen Pappa, Reading RG6 1PT (GB)

Correspondence Address: KLAUBER & JACKSON, 411 HACKENSACK AVENUE, HACKENSACK, NJ, 07601, US

(21) Appl. No.: 10/487,163

(22) PCT Filed: Aug. 16, 2002
(86) PCT No.: PCT/GB02/03816

(30) Foreign Application Priority Data
Aug. 17, 2001 (GB) 0120147.4

(51) Int. Cl. A61K 31/56; A61K 31/501; A61K 31/473

(52) U.S. Cl. 514/171; 514/284; 514/252.05

(57) ABSTRACT

The invention relates to a method for the treatment of an oestrogen-dependent proliferative disorder of the uterus such as endometriosis and uterine fibroids in a patient, by administering an aromatase inhibitor to the patient intravaginally. This achieves high local levels of aromatase inhibitor Locally, and therefore avoids some of the adverse reactions that are observed when aromatase inhibitors are delivered orally. Further, intravaginal delivery allows inhibition of the local lesional production without significantly affecting the circulating levels which have been produced by the ovaries. This results in minimal side-effects and will allow for longer term treatment than current therapies.

The invention concerns a method for the treatment of an oestrogen-dependent proliferative disorder of the uterus in a patient, by administering an aromatase inhibitor to the patient intravaginally.

All publications, patents and patent applications cited herein are incorporated in full by reference.

The incidence of proliferative disorders of the uterus is very high in women of child-bearing age. In particular, conditions known as endometriosis and uterine fibroids affect up to 1 in 4 of women in the Western world.

Endometriosis is a benign (non-cancerous), chronic condition affecting women. It is defined as the presence of both endometrial glandular tissue and stromal cells located outside the uterus. This displaced tissue is known as ectopic endometrium. This disease affects women during their childbearing years with deleterious social, sexual and reproductive consequences. Endometriosis has been proposed as one of the most commonly-encountered diseases of gynaecology, with the incidence of endometriosis in the general population being estimated to be around 5%, although it is thought that it affects 10% to 15% of women between the ages of 20 and 45 years. In North America it is estimated that 5.5 million women suffer with the condition. In Japan a large survey conducted by the Ministry of Health in 1997 found that around 130,000 women are undergoing hospital treatment for the condition at any one time, and that the incidence of endometriosis was higher than that for cancer and diabetes. In the United Kingdom, endometriosis causes the loss of 45 days work each year, on average, for every patient and 11% of sufferers claim state benefit as a direct result of the condition. (Data Sources: Pharma Ventures, Eurotech Capital and Bridgehead Technologies)

Endometriosis develops over a number of years until the symptoms are sufficiently severe for the affected patient to seek medical advice. The condition is diagnosed by using laparoscopy to visualise the endometriotic deposits. The most common and severe symptom is chronic pelvic pain, as well as dysmenorrhoea (pain in the back and lower abdomen during periods) and dyspareunia (pain during intercourse). A link between endometriosis and infertility is evident and between 30% and 40% of women with endometriosis are infertile, although the mechanistic link has not been clarified.

The development of endometriosis involves the establishment and growth of endometrial cells at ectopic sites, most commonly the pelvic peritoneum, ovaries and rectovaginal septum, following retrograde menstruation (see Thomas & Prentice (1992), Repro. Med. Rev., (1): 21-36, and Sampson, 1927, Am. J. Obstet. Gynecol., 14: 422469). The sustained growth of the endometriotic lesions depends on the formation of new blood vessels. Indeed, angiogenic factors, including vascular endothelial growth factor (VEGF), have been detected in the peritoneal fluid of women with endometriosis (see, for example, Oosterlynck, et al., 1993, Fertil. Steril., 59: 778-782; European patent application EP-A-0 771 192; McLaren, et al., 1996, Hum. Reprod., 11: 200-223; and Nisolle. & Donez, 1997, Fertil. Steril., 68,575-596).

Implantation of autologous non-malignant ectopic tissue is a unique phenomenon suggesting that an abnormal host response may be present in women who develop this disease. This theory is supported by the fact that only a minority of women will develop the disease in spite of the common occurrence of retrograde menstruation as a source of endometrial tissue.

Endometriosis is now recognised as a multifactorial disease caused by genetic factors that may determine a woman's susceptibility to endometriosis as well as environmental factors. It has been reported that first degree relatives of an affected individual have an increased risk of developing endometriosis compared to the general population (see, for example, Kennedy et al., 1995, J. Assist. Reprod. Genetic., 12(1): 32-34; Treolar et al., 1999, Fertil. Steril., 71(4): 701-710; and Malinal et al., 1980, Am. J. Obstet. Gynecol., 137(3): 332-337).

There are many theories proposed for the origin of the disease and various cellular and biochemical constituents of the peritoneal fluid have been reported to play an important role in the pathogenesis of this disease. Alterations in multiple aspects of both humoral immunity and cell-mediated immunity have also been reported in suffering individuals (Understanding and Managing Endometriosis. Advances in Research and Practise Ed. A. Lemay & R. Maheux Parthenon Publishing London New York 1999).

The growth and development of endometrial tissue appears to depend on the presence of oestrogen. Drugs that reduce the effect of GnRH (both agonists and antagonists have this effect) reduce oestrogen levels as well as levels of progesterones. These drugs, developed initially to treat oestrogen-dependent tumours provide some symptomatic relief in endometriosis. Pregnancy and some oral contraceptive agents also bring about symptomatic relief. Furthermore, all drugs described to date act by relieving the symptoms of the disease and are not in any sense curative. This makes a patient permanently dependent on the drug if the symptoms of disease are to be kept at bay.

Presently, the only treatment of endometriosis that is effective in the long term involves surgery, although recurrence of the disease often occurs within 5 years post-operation. Certain developments in the treatment of endometriosis, such as the identification of angiogenic growth factors (such as VEGF) which are thought to be partly responsible for the establishment and development of endometriosis, have in part paved the way for the development of new therapeutic agents. Additionally, the discovery of aromatase expression from endometriotic lesions has been suggestive of the use of aromatase inhibitors to treat endometriosis (Bulun et al., 1999, Endocrine Related Cancer, 6: 293-301). Indeed, one unusually aggressive case of postmenopausal endometriosis has been successfully treated with an aromatase inhibitor (Bulun et al., 2000, Hum Reprod Update, 6(5):413-8). However, to proceed beyond this initial work, clinical trials will clearly be required to establish whether aromatase inhibitors will have a significant role in the medical management of endometriosis.

Uterine fibroids (also known as leiomyomas, fibromyomas, fibromas, myofibromas and myomas; these terms may be used interchangeably) are a second form of proliferative disorder of the uterus that afflict a large number of women. These benign growths, deriving from the myometrium, consist of masses of smooth muscle fibres and white fibrous tissues which can range in size from a small seed to a mass weighing as much as a kilogram. They occur in or on the surface of the uterus, and affect around 20%-40% of women, generally between the ages of 35 and 45 years. In the United States, Europe and Japan an estimated 3.4 million women are diagnosed each year with fibroids, of whom a significant number receive therapy. According to the Society of Cardiovascular and Interventional Radiology in the USA, 20%-40% of women have fibroids of a significant size and among African-American women the incidence is as high as 50%.

Many women with uterine fibroids are asymptomatic, although many experience abnormal uterine bleeding (menorrhagia), pelvic pain, pelvic pressure, urinary frequency or compressive bowel symptoms. Some women appear to have difficulty becoming pregnant, or/and have an increased risk of miscarriage.

The cause of uterine fibroids is not known, but once formed they establish a network of new blood vessels that provides the blood supply to allow them to increase in size.

Uterine fibroids may be treated by conservative surgery, such as endometrial resection of submucous fibroids during hysteroscopy, or by more invasive surgery such as myomectomy (the surgical removal of the fibroid) and hysterectomy. In the USA, more than 200,000 women undergo hysterectomy each year due to fibroids. Less invasive kinds of surgery are available to remove small fibroids, like hysteroscopy, when a fibre optic scope is inserted through the vagina to remove them. However, about 10% of women will require repeat surgery as the fibroids re-grow.

Medical treatments of fibroids make use of a variety of agents that may reduce the symptoms of pain and heavy blood loss as well as contraceptives and progestogens. A number of women use GnRH agonists to shrink the fibroid but this treatment can only be useful for up to six months and fibroids may re-grow after treatment.

There thus also remains a great need for the discovery of agents with effective prophylactic or therapeutic value against proliferative disorders of the uterus, such as endometriosis and uterine fibroids.

### SUMMARY OF THE INVENTION

According to the invention, there is provided a method of treating or preventing an oestrogen-dependent proliferative disorder of the uterus in a patient, by administering an aromatase inhibitor to the patient intravaginally.

The inventors have discovered that the vagina provides an excellent route for the administration of an aromatase inhibitor to treat or prevent the incidence of oestrogen-dependent proliferative disorders of the uterus such as endometriosis and uterine fibroids.

It has been found, through biopsy of endometrial lesions and uterine fibroids, followed by the specific amplification of nucleic acids that encode aromatase by performing quantitative PCR (QPCR), that significantly elevated levels of aromatase are expressed in endometrial lesions and fibroids. Aromatase activity is barely detectable in normal (eutopic) endometrium and myometrium.

This inappropriate aromatase activity gives rise to local biosynthesis of oestrogen. Upregulation of VEGF results from increased oestrogen. This favours the accumulation of oestrogen and VEGF in endometriotic tissues, which may contribute to the establishment and growth of vasculature of the lesions through oestrogen-induced VEGF production.

Additionally, oestrogen-dependent induction of cyclooxygenase gives rise to elevated concentration of prostaglandin E₂ (PGE₂). PGE₂ has been shown to be a potent stimulator of aromatase expression in endometriotic stromal cells (Noble, L. S. et al. 1997 J. Clin. Endocrinol. Metab. 82 No. 2 600-606). Thus, a positive feedback loop is formed and this favours continuous production of oestrogen in the endometriotic tissues. Aromatase inhibitors are considered to break this cycle by blocking local production of oestrogen in endometriosis. However, these inhibitors also decrease or block oestrogen formation in other tissues, such as the ovary and subcutaneous fat.

The increased levels of PGE₂ may be responsible for some of the symptoms of endometriosis, such as pain. Inhibition of oestrogen production with an aromatase inhibitor has been noted by Bulun et al to result in a reduction in the size of an endometrial lesion in one patient (Takayama, K. et al 1998 Fertil. Steril. 69 No. 4 709-713).

Intrinsic molecular aberrations in pelvic endometriotic implants have been previously proposed to contribute significantly to development of endometriosis. For example, aberrant expression of aromatase, certain cytokines and tissue metalloproteinases, deficiency of 17β-hydroxysteroid dehydrogenase (17β-HSD) type 2 and resistance to the protective action of progesterone are some of these molecular abnormalities (see Khorram et al., 1993, American Journal of Obstetrics and Gynecology, 169: 1545-1549; Sharpe-Timms et al., 1995, Journal of Clinical Endocrinology and Metabolism, 80: 3784-3787; Noble et al., 1996, Journal of Clinical Endocrinology and Metabolism, 81: 174-179; Osteen et al., 1996, Seminars in Reproductive Endocrinology, 15: 301-308; Bruner et al., 1997, Journal of Clinical Investigation, 99: 2851-2857; Zeitoun et al., 1998, Journal of Clinical Endocrinology and Metabolism, 83: 4474-4480; and Zeitoun et al., 1999, Molecular Endocrinology, 13: 239-253).

A number of studies have demonstrated that oestrogen is produced in endometriotic implants in the pelvis and at other sites through the expression of the aromatase enzyme, (see Takayama and Bulun, 2001, Nippon Rinsho; 59 Suppl 1:157-60; Kudoh et al., 1997, J Steroid Biochem Mol Biol; 63(1-3); 75-80; Bulun et al., 2000, Hum Reprod Update; 6(5): 413-8; Kitawaki et al., 2000, J Clin Endocrinol Metab; 85(9): 3292-6; Bulun et al., 2000, J Mol Endocrinol; 25(1):3542; Bulun et al., 1999, Endocr Relat Cancer; 6(2): 293-301; Bulun et al., 2000, Trends Endocrinol Metab; 11(1): 22-7; Zeitoun et al., 1999, Fertil Steril; 72(6): 961-9; Zeitoun et al., 1999, Mol Endocrinol; 13(2): 239-53; Takayama et al., 1998 Fertil Steril; 69(4): 709-13; and Bulun et al., 1997, J Steroid Biochem Mol Biol; 61(3-6): 133-9). Prostaglandins in endometriotic cells have been proposed to stimulate aromatase activity to increase oestrogen biosynthesis. In turn, oestrogen stimulates prostaglandin formation and the growth of endometriotic implants.

It is also known that endometrial lesions are oestrogen-dependent. During the earlier stages of the development of the lesions, the oestrogen is supplied by the systemic circulation and the source of the hormone is predominantly the ovaries; a small and variable amount of oestrogen may be produced by adipose tissue. Growth of lesions is thought to relatively slow during the early stages but later a number of cellular and molecular changes have been documented. Of particular significance is the local lesional production of oestrogen. This occurs when the mesenchymal elements of endometrial lesions begin the aberrant synthesis of certain enzymes. The local production of oestrogen stimulates growth of glandular tissue and also promotes synthesis of pain-mediators, especially the prostaglandins. With time, the local production of oestrogen becomes more significant than ovarian-synthesized circulating hormone.

It is well established that reduction in ovarian production of oestrogen ameliorates the symptoms of endometriosis. However the current methods for achieving this involve very significant ovarian suppression to circulating levels where a number of oestrogen-dependent organs become compromised. For example, bone mineral density is critically dependent on adequate levels of oestrogen. For this and other reasons, treatment with agents such as the gonadotrophin-releasing hormone agonists can only be used for six months.

The inventors have now recognized that a significant proportion of existing endometrial lesions may be targeted by the transvaginal delivery of an aromatase inhibitor. This achieves high local levels of aromatase inhibitor locally, and therefore avoids some of the adverse reactions that are observed when aromatase inhibitors are delivered orally. By transvaginal delivery is meant the movement of aromatase inhibitor molecules from the vaginal cavity into the surrounding tissues via a simple diffusion process.

The invention exploits the highly vascularised nature of the vaginal mucosal tissue (which leads to a copious blood supply) to deliver an aromatase inhibitor composition to localised areas and the underlying tissues that are diseased. Delivery of the aromatase inhibitor through the tissue wall is thus fast and this route of administration facilitates achieving a concentration of drug that is effective to treat or prevent the disorder. A significant effect of the invention is that intravaginal delivery allows inhibition of the local lesional production without significantly affecting the circulating levels which have been produced by the ovaries. This results in minimal side-effects and will allow for longer term treatment than current therapies. The lowering of the locally high levels of oestrogen will reduce growth of lesions and also lower the rates of production of inflammatory mediators which lead to the major symptom of pain.

The vasculature of the female reproductive tract consists of a number of vascular plexuses of different origin, and there are anastomoses between a number of the key vessels including the vaginal, ovarian and uterine arteries as well as between the uterine and deep perineal branches of the pudendal artery. For example, the arterial supply to the vagina is from the vaginal branch of the uterine artery, occasional vaginal branches of the internal iliac arteries, possible twigs from the middle rectal arteries, and branches from the internal pudendal arteries. These vaginal arteries course along the lateral vaginal walls, along the walls of the uterus, and anastomose with the ovarian artery. The ascending branches of the uterine artery lead to the tubal arterial branch and anastomoses with the ovarian artery. Venous return from the vaginal venous plexus drains into the internal iliac vein, and the uterine venous return is along uterine veins, which generally parallel the arterial supply. Lymphatic drainage of the vagina drains into the external and internal iliac nodes, as well as into the superficial inguinal nodes. Lymphatic drainage of the uterus parallels the uterine blood supply.

The absorption of drugs through the vaginal mucosa and into the systemic vasculature is known, and it has been observed by others that a “uterine first pass effect” occurs whereby, for example, the ovarian and uterine tissue levels of some drugs given vaginally are higher than would be expected from oral administration of the same doses. The exact mechanism of this “first uterine pass” effect is not yet fully understood. Four hypotheses can be put forth to explain the uterine first pass effect: (1) transvaginally administered drugs may transit to the uterus and other local tissues through the local circulatory system; (2) there may be direct diffusion of drug into the uterus and other local tissues; (3) drugs may reach the uterus through the lymphatics; or (4) a “counter-current” redistribution of drug may occur between arteries and veins.

By an “oestrogen-dependent proliferative disorder of the uterus” is meant any oestrogen-dependent non-malignant disorder that occurs in females that stems from uterine tissue. Particular examples of oestrogen-dependent proliferative disorders of the uterus that are included within the terms of the invention are endometriosis and uterine fibroids. Humans are preferred patients for treatment, although non-human mammals, such as domesticated and companion animals, may also be treated.

Endometriosis is the name given for the occurrence of endometrium tissue, found at ectopic sites in the body. Endometriotic lesions may be determined histologically using markers or by looking for endometrial glands and stromal elements in tissue at ectopic sites. Although this tissue type may be in any anatomical location, it is generally to be found in the region of the ovary, peritoneum, or recto-vagina and it is endometriotic lesions in these locations that may be particularly beneficially treated according to the method of the present invention.

Uterine fibroids, or uterine leiomyomas are a second oestrogen-dependent proliferative disorder of the uterus, marked by the presence of one or more benign tumours found consisting of connective tissue and muscle found on the uterine wall. Fibroids may lie just below the uterine lining or near the uterus' outer covering, whilst others are located near the cervix, or close to the openings of the fallopian tubes. They are usually detected on abdominal and pelvic examination as well as on internal examination, which may reveal the uterus to be enlarged and/or detect the fibroid as a smooth, firm lump. Ultrasound is the most useful test for diagnosing fibroids as these tumours have a characteristic appearance that distinguishes them from pelvic cysts.

According to the invention, an aromatase inhibitor should be delivered intravaginally. By “intravaginally” is meant that an aromatase inhibitor is administered via the vagina, such that the inhibitor crosses the vaginal mucosa to enter the blood and/or lymphatic system by way of local absorption though the highly vascularised tissue in this area.

Vaginal administration of an aromatase inhibitor for the treatment of endometriosis and fibroids is anticipated to lead to both improved efficacy, and to a reduced adverse effects profile compared to oral therapy.

Of great significance is the fact that an effective concentration of aromatase inhibitor may effectively be delivered to the diseased tissue itself, i.e. the endometrial lesion or uterine fibroid, both of which disorders generally present in tissues or organs that are close to the vagina, such as in the vaginal tissues, uterus, ovaries and fallopian tubes, rectovaginal region and cul-de-sac and other tissues and organs within the peritoneal cavity. In addition, such levels of aromatase inhibitor are expected to affect newly-shed endometrial cells travelling up the fallopian tubes and into the peritoneum, reducing their rate of growth and the probability of implantation.

The oral delivery of a drug such as an aromatase inhibitor may involve the destruction of the aromatase inhibitor by local conditions such as those that are encountered in the stomach. First-pass metabolism by the liver is also a problem suffered by the oral delivery route. Furthermore, in order to achieve a concentration of aromatase inhibitor that is effective at the diseased tissue, a large dose must be administered that then leads to high systemic levels of inhibitor compound. The oral use of aromatases to treat metastatic breast cancer has been associated with a variety of adverse events including hot flushes, dizziness, oedema, sweating, nausea, vomiting, chest or back pain, fatigue, headache, insomnia, dyspnoea, asthenia, emotional lability, and depression.

By administering the aromatase inhibitor via the vaginal route, a much lower concentration of inhibitor compound needs to be used than would be necessary if the inhibitor were to be administered parenterally, for example, by the oral route. The method of the invention yields significant local levels of aromatase inhibitor while maintaining circulating blood levels that are low enough to avoid most undesired side effects. This means that susceptible tissues will be exposed to a much lower concentration of inhibitor, whilst diseased areas will be exposed to a much higher concentration of inhibitor than would be achieved using any other method of administration.

The oral route is also not suitable for certain types of drug compound—in order to be effectively absorbed into the blood, an orally-delivered drug compound must be orally-active, which means that it is either passively transported or actively transported. Only a small proportion of drug molecules fulfil all these criteria, which thus limits the number of drug compounds that might be used to treat an oestrogen-dependent proliferative disorder of the uterus. The oral route is also precluded when vomiting has occurred, or is likely to occur, or when the patient is unable to swallow successfully.

Intra-vaginal devices are known that are suitable for the delivery of aromatase inhibitors intravaginally, according to the method of the invention. Suitable devices include those of the type where a medicament is impregnated into the device, and of the type that carries an encapsulated medicament.

Movement of pharmaceutical molecules from the vaginal cavity into the surrounding tissues will generally occur via a simple diffusion process. Net diffusion may be given by the equation:
Net diffusion=k.D.(C_{vag}−Cₜᵢₛₛ);
where k is a constant, D is the diffusion constant for the molecule, C_{vag} is the molecular concentration in the vagina at the surface of the mucosa and Cₜᵢₛₛ is the molecular concentration in the tissue surrounding the vagina. In order to achieve maximum rates of uptake of pharmaceuticals from the vaginal cavity across the vaginal mucosa and into the surrounding tissues and body fluids, C_{vag} should be maintained at as high a level as possible.

Examples of devices suitable for the intravaginal delivery of an aromatase inhibitor include those described in U.S. Pat. No. 4,309,997, U.S. Pat. No. 4,318,405, U.S. Pat. No. 5,273,521, U.S. Pat. No. 5,299,581, GB 1,581,474, U.S. Pat. No. 5,954,688, U.S. Pat. No. 4,402,693, U.S. Pat. No. 3,948,265, U.S. Pat. No. 6,086,909, U.S. Pat. No. 3,545,439, U.S. Pat. No. 3,902,493, U.S. Pat. No. 2,739,593, U.S. Pat. No. 3,521,637, U.S. Pat. No. 3,884,233, U.S. Pat. No. 4,286,596, U.S. Pat. No. 6,197,327, U.S. Pat. No. 5,527,534, EP-A-0,703,802, GB 2,069,336, U.S. Pat. No. 4,601,714, U.S. Pat. No. 5,299,581, U.S. Pat. No. 6,159,174, WO99/18884, WO99/40966, W000/48539 and co-owned, co-pending International patent application PCT/GB01/01789. Other examples of suitable devices will be clear to those of skill in the art.

The aromatase inhibitor may also be administered intravaginally as a bioadhesive formulation, for example, in the form of a gel, cream, tablet, pill, capsule, suppository, film, or any other pharmaceutically acceptable form that remains in the vaginal cavity and does not wash away easily. If applied as a bioadhesive formulation, the formulation should remain attached to the epithelial surfaces of the vaginal mucosa for a significant period, at the minimum, for example, between about 30 minutes to twenty-four hours. This preferred level of bioadhesion may advantageously be attained by the inclusion of a bioadhesive agent in the pharmaceutical formulation, such as a cross-linking agent, so that an appropriate level of bioadhesion results. Suitable formulations are described, for example, in U.S. Pat. No. 4,615,697.

Suitable aromatase inhibitors for use in the methods of the present invention include any compound that inhibits the formation of oestrogens from their precursors by an aromatase enzyme. Such compounds can be used either alone or in combination with other aromatase inhibitor compounds. Certain classes of suitable aromatase inhibitors include non-steroidal, weak steroid and steroidal aromatase inhibitors.

Examples of aromatase inhibitors that are suitable for use in the present invention include anastrozole (Armimidex); letrozole; exemestane; vorozole; YM 511 (Yamanouchi Pharmaceutical); YM 553 (Yamanouchi Pharmaceutical); [(4-bromobenzyl)(4-cyanophenyl)amino]azoles and their azine analogs; 4-N-substituted amino-4H-1,2,4-triazole derivatives; 3-[N-(2-chlorobenzyl)amino]-6-(1H-imidazol-1-yl)pyridazine dihydrochloride (CAS 124070-28-3, MFI-279); aminoglutethimide; 4-hydroxy-androstenedione; 4-hydroxy-4 -androstene-3,17-dione; 4-acetoxy-4-androstene-3,17-dione; fadrozole hydrochloride (CGS 16949A) (Bonzol; Mitsubishi-Tokyo Pharmaceuticals Inc); formestane; 1-methylandrosta-1,4-diene-3,17-dione; 1-methylandrosta-1,4-diene-3,17-dione (described in German patent application 3,322,285); testolactone (17a-oxa-D-homoandrosta-0,1,4-diene-3,17-dione) (described in “Journal of Clinical Endocrinology and Metabolism”, (1979) 49: 672); androsta-4,6-diene-3,17-dione; androsta-4,6-dien-17.beta.-ol-3-one acetate; androsta-1,4,6-triene-3,17-dione; 4-androstene-19-chloro-3,17-dione; 4-androstene-3,6,17-trione; compounds described in “Endocrinology” (1973), vol. 92(3): 874; the 19-alkynylated steroids disclosed in German patent application 3,124,780; the 10-(1,2-propadienyl) steroids described in German patent application 3,124,719; the 19-thioandrostane derivatives described in EP-A-0,100,566; 4-androsten4-ol-3,17-dione (described in “Endocrinology” 1977, 100(6): 1684 and in U.S. Pat. No. 4,235,893), and its esters; the 1-methyl-15.alpha.-alkyl-androsta-1,4-diene-3,17-diones described in German patent application 3,539,244; the 10.beta.-alkynyl-4,9(11)-estradiene derivatives described in German patent application 3,644,358; 1,2.beta.-methylene-6-methylene-4-androstene-3,17-dione (described in EP-A-0 250 262); or mixtures of any of these aromatase inhibitor compounds.

A number of references detail the effect of aromatase inhibitors such as those listed above in inhibiting oestrogen production, including the following: Kudoh et al., 1997, J Steroid Biochem Mol Biol, 63(1-3): 75-80; Okada et al., 1997, Chem Pharm Bull (Tokyo), 45(8): 1293-9; Okada et al., 1997, Chem Pharm Bull (Tokyo), 45(3): 482-6; Okada et al., 1996, Chem Pharm Bull (Tokyo), 44(10):1871-9; Kudoh et al., 1996, J Steroid Biochem Mol Biol, 58(2): 189-94; Kudoh et al., 1995, J Steroid Biochem Mol Biol, 54(5-6): 265-71.

The dosage of the aromatase inhibitor that is administered should be therapeutically-effective, i.e. a dosage amount that is necessary to treat, ameliorate, or prevent the oestrogen-dependent proliferative disorder of the uterus, or to exhibit a detectable therapeutic or preventative effect. This dosage will vary depending on various factors such as the potency of the inhibitor compound, its toxicity in the treated patient, the general health, age and weight of the patient, the hormonal levels of the patient, the patient's diet, the time and frequency of administration, drug combination(s), reaction sensitivities, tolerance/response to therapy, the stage of the disease, the degree of spread of diseased tissue, the lifetime of the inhibitor compound in its active state, the solubility of the compound, its absorption characteristics across the vaginal mucosa, the eventual tissue concentration to be attained and so on. [This dosage amount can be determined by routine experimentation and is within the judgement of the clinician.]

For any aromatase inhibitor compound, a therapeutically effective dosage can be estimated initially either in cell culture assays, for example, endometrial or smooth muscle cells from fibroids of neoplastic cells, or in animal models, usually mice, rabbits, dogs, or pigs, primates such as baboons, macaques, and so on. The animal model may also be used to determine the appropriate concentration range for administration. Such information can then be used to determine useful dosages for humans.

Generally, the amount administered in one dose will be between 100 μg and 1 g, preferably between 100 μg and 10 mg of an aromatase inhibitor such as 1-methylandrosta-1,4-diene-3,17-dione, or a biologically-equivalent dose of any other aromatase inhibitor as listed above. The amount selected will depend, of course, on the dosage prescribed, but undesirably high dosages may advantageously be avoided by using intravaginal delivery, as the concentration of inhibitor compound in the vicinity of the vaginal tissue wall is maintained at a high level.

The frequency of dosage may also be varied so as to administer an aromatase inhibitor most effectively. Conveniently, the dose may be repeated either daily, weekly, monthly, or quarterly (three monthly). Of course, the actual amount that is administered will be altered to take the dosage frequency into account.

In order to be administered intravaginally, the aromatase inhibitor should preferably be administered as part of a pharmaceutical formulation, including a pharmaceutically-acceptable carrier. Such carriers include large, slowly metabolised macromolecules such as proteins, polysaccharides, polylactic acids, polyglycolic acids, polymeric amino acids, amino acid copolymers, polyethylene glycol, PDMS, microspheres, hydrogels, and inactive virus particles, provided that the carrier does not itself induce the production of antibodies harmful to the individual receiving the composition, and which may be administered without undue toxicity. Pharmaceutically acceptable salts can also be used, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in the pharmaceutical formulations of the invention may additionally contain liquids such as water, saline, glycerol and ethanol. Additionally, auxiliary substances, such as wetting or emulsifying agents, pH buffering substances, lubricants, plasticizing agents, preservatives, gel formers, tablet formers, pill formers, suppository formers, film formers, cream formers, disintegrating agents, coatings, binders, vehicles, colouring agents, taste and/or odour controlling agents, humectants, viscosity controlling agents, pH-adjusting agents, absorption enhancers, and the like, may be present in such compositions.

According to a further aspect of the invention, there is provided a pharmaceutical formulation comprising an aromatase inhibitor compound, for use in the treatment of an oestrogen-dependent proliferative disorder of the uterus by intravaginal administration.

The invention also provides the use of an aromatase inhibitor compound in the manufacture of a medicament for the treatment of an oestrogen-dependent proliferative disorder of the uterus by intravaginal administration.

The invention also provides an intra-vaginal device comprising an aromatase inhibitor compound according to any one of the embodiments of the invention described above, in a therapeutically-effective amount. The inhibitor compound may be coated onto the intra-vaginal device, impregnated or absorbed into the device, or applied to the device by any suitable means that allows the compound to be attached or bonded to the device, yet which allows the compound to be available for absorption into the vaginal mucosa, as will be clear to those of skill in the art. A particular preferred example of a suitable intra-vaginal device is that described in co-pending, co-owned International patent application PCT/GB01/01789.

Various aspects and embodiments of the present invention will now be described in more detail by way of example. It will be appreciated that modification of detail may be made without departing from the scope of the invention.

### BRIEF DESCRIPTION OF THE FIGURES

FIG. 1A-C: Comparison of aromatase transcript levels in human eutopic and ectopic endometrium in 11 patients suffering from endometriosis. Aromatase levels of eutopic endometrium are shown by open bars and the levels in endometriotic lesions by filled bars. Table 1 provides an explanation of the nomenclature used in these Figures.

FIG. 2: Comparison of aromatase transcript in human myometrium and uterine fibroid tissue (QPCR TaqMan data).

FIG. 3 shows levels of radioactive counts in endometrial lesions in a baboon model following intra-vaginal administration of a radiolabelled aromatase inhibitor.

FIG. 4: A: Laparoscopic aspect of lesion 5 in the medial anterior bladder of baboon PAN2615. B: Laparoscopic aspect of lesion 10 in the posterior uterus of baboon PAN2615.

FIG. 5: A. and B: Histological confirmation of the presence of endometrial glands in lesion 13 of baboon PAN2615. C: Histological confirmation of the presence of glands in eutopic endometrium of baboon PAN2615. The presence of spiral arterioles is also shown.

### EXAMPLE 1

### Detection of Aromatase Transcript in Human Myometrium and Uterine Fibroid Tissue

RNA isolation from fresh tissues was performed using the TRIZOL Reagent Method for the myometrial and fibroid samples and the QIAGEN Midi RNeasy kit for all endometrial samples. For the myometrial and fibroid samples, 1 μg of RNA was reverse-transcribed to cDNA using the Superscipt First Strand Synthesis Kit (GIBCO BRL, Life Technologies) and random primers. 0.1 and 2 μg of RNA were used for reverse transcription of the endometrial samples. The cDNA quality was validated by amplification of the GAPDH gene and the absence of genomic DNA was confirmed by using calbindin specific primers in exon 1 and 2 of the calbindin gene. (CALB-FOR: GACACACACCCCGCTGTAC; CALB-REV: TGCTGGAGCTCCTGGATC)

For the endometrial and endometriosis samples, undiluted cDNA was used for the Q-PCR experiments, while 1:10 dilution of cDNA was used for all the myometrial and fibroid samples.

Primers and a probe specific for the aromatase gene were designed using the Primer Express Software. The probe was labelled with FAM dye (Xmax=518 nm). The sequences of primers and the probe are shown below:

| | |
|---|---|
| Aromatase Forward | CCCCGGCCTTGTTCGT |
| Aromatase Reverse | CCTCCAACCTGTCCAGATGTGT |
| Aromatase Probe | TGGTCACAGTCTGTGCTGAATCCCTCAA |

The primer concentrations were optimised in order to determine the minimum primer concentration for obtaining maximum signal. The PCR reaction mix used for primer optimisation consisted of 25 μl of TaqMan Universal PCR Master Mix (2×), 5 μl of Forward primer (50-900 nM), 5 μl of Reverse primer (50-900 nM), 5 μl of 2 μM TaqMan probe, 5 μl of DNA sample and 5 μl of water. The thermal cycling conditions for primer and probe optimisation are described below:

| | | |
|---|---|---|
| 50° C. | 2 min | 1 cycle |
| 95° C. | 10 min | 1 cycle |
| 95° C. | 15 sec | 40 cycles |
| 60° C. | 1 min | |

The probe concentration was optimised by using the conditions described for the primer optimisation, where the forward and reverse primers are used at their optimal concentrations.

All assays were then performed using optimum primer and probe concentrations and thermal cycling conditions shown below:

PCR reaction mix

| | |
|---|---|
| AmpliTaq Master Mix | 12.5 μl |
| Forward primer | 300 nM |
| Reverse primer | 300 nM |
| Probe (1 μM stock) | 2.5 μl |

Thermocycling conditions

| | | |
|---|---|---|
| 50° C. | 2 min | 1 cycle |
| 95° C. | 10 min | 1 cycle |
| 95° C. | 15 sec | 35 cycles |
| 60° C. | 1 min | |

All experiments were performed in triplicate and all samples were normalised by using as endogenous control, 18S mRNA, to account for differences in the amount of total RNA added to each reaction. The 18S Q-PCR was performed using the exact same dilution and quantity of cDNA as used for the aromatase Q-PCR and following the protocol described in the PE Biosystem's pre-developed assay. Human placenta RNA was used as the reference sample for allowing comparisons between different samples.

Two transcript variants of aromatase exist, variant 1 (GeneBank entry NM-000103) and variant 2 (GeneBank entry NM-031226) which has a longer 5′ UTR than variant 1. Both variants encode the same protein. The PCR primers used in this invention were specific for regions common to both variants.

Raised levels of aromatase transcript were observed in the endometriotic lesions of all 11 endometriosis patients tested. The relative amount of aromatase transcript was patient-dependent with endometriotic lesions from some patients having as much as several thousand-fold higher levels when compared to the eutopic endometrium (H) from the same patient (such as in patients S and Q). Aromatase transcript was barely detectable in the eutopic endometrium of all patients tested.

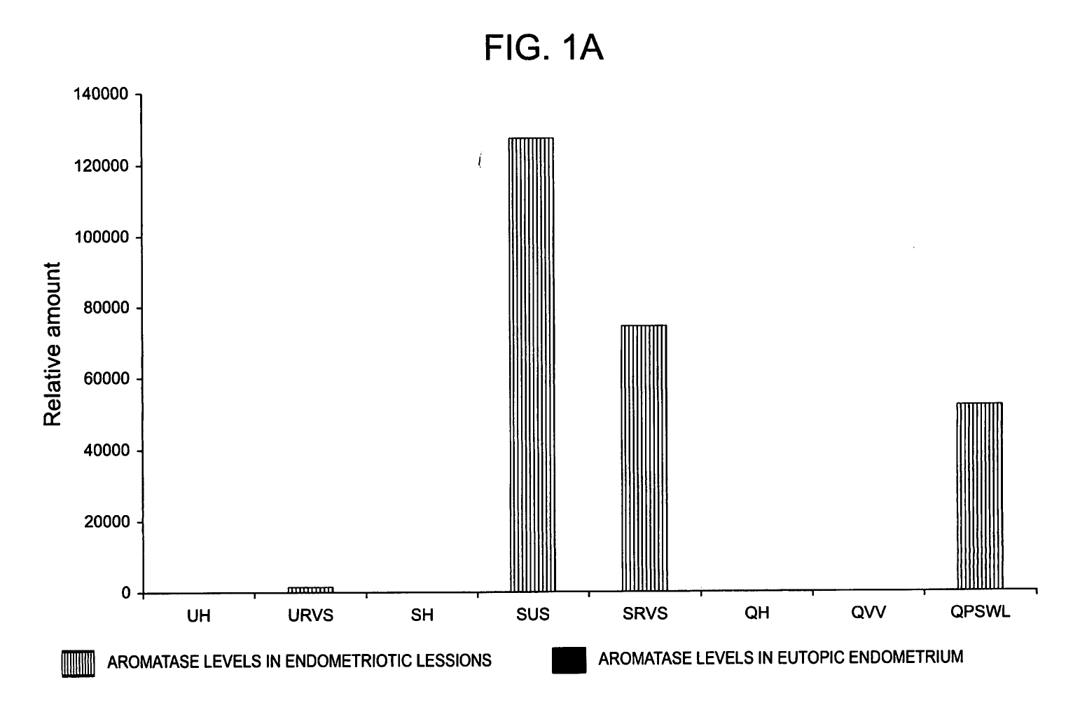

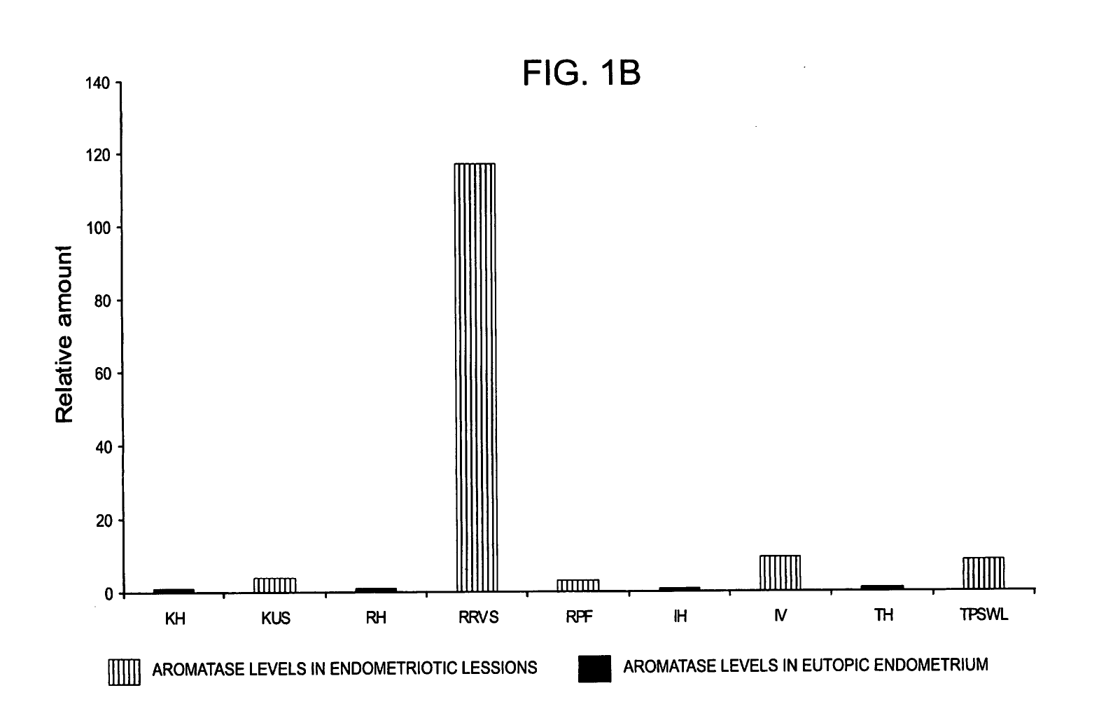

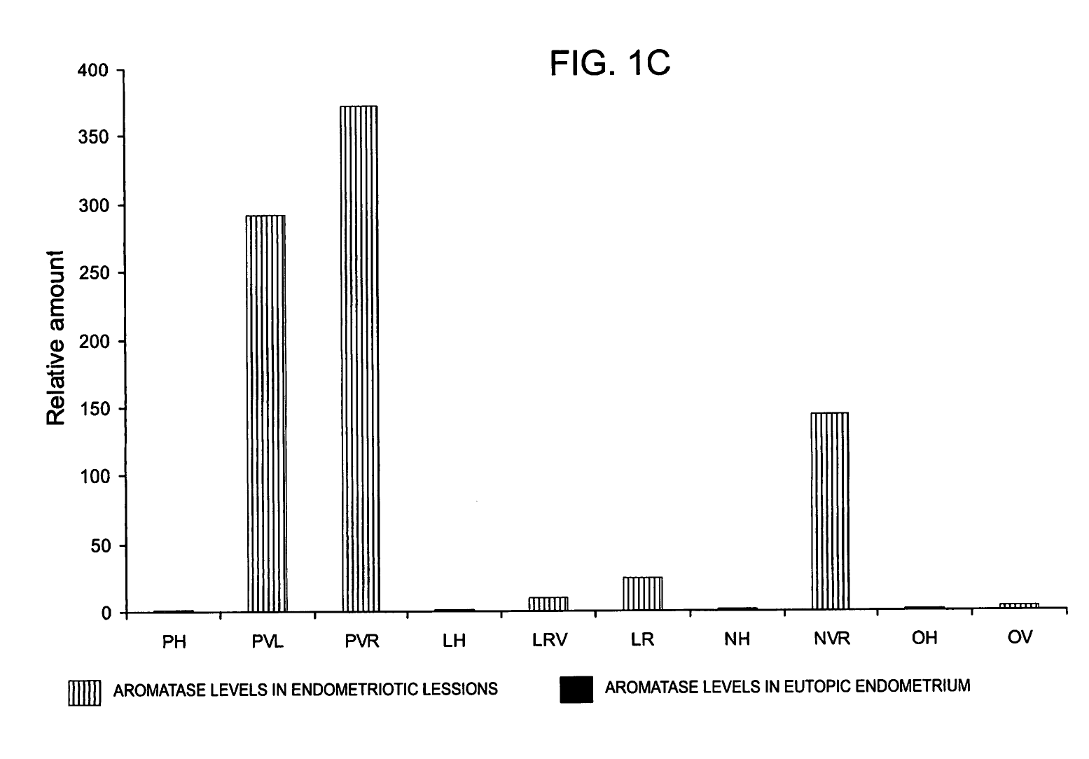

Most interestingly, aromatase transcript was upregulated in lesions located at different regions of the patient's peritoneal cavity whether they were found on uterosacral ligament, (SU) retovaginal septum (RFS), ovaries (V) or/and pelvic side wall (PSWL) (FIG. 1).

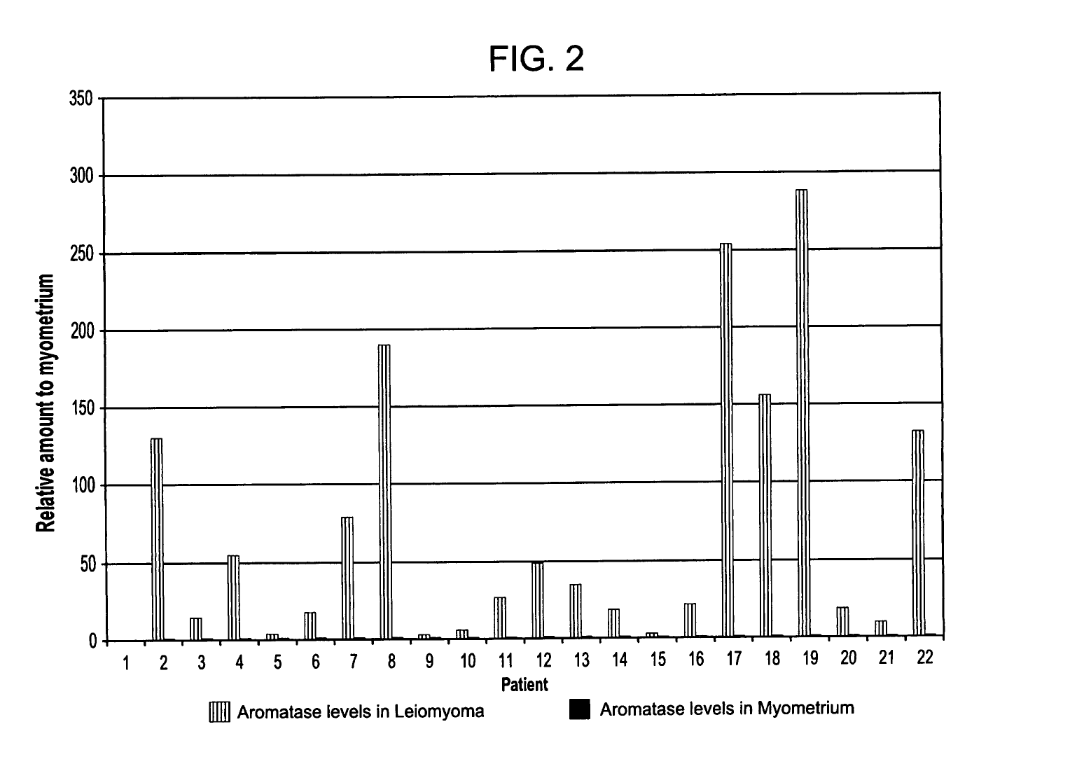

High levels of aromatase transcript were also observed in all fibroid specimens tested. Aromatase was barely detectable in matched myometrium from the same patients. 40% of the fibroids tested had levels higher than 50-fold when compared to their corresponding myometrium (FIG. 2).

TABLE 1
Nomenclature used in FIGS. 1 A-C.

| | |
|---|---|
| UH | Patient U, eutopic (healthy) endometrium |
| URVS | Patient U, endometriotic lesion isolated from |
| | rectovaginal septum |
| SH | Patient S, eutopic (healthy) endometrium |
| SRVS | Patient S, endometriotic lesion isolated from |
| | rectovaginal septum |
| QH | Patient Q, eutopic (healthy) endometrium |
| QVV | Patient Q, endometriotic lesion isolated from vaginal vault |
| QPSWL | Patient Q, endometriotic lesion isolated from pelvic side wall |
| KH | Patient K, eutopic (healthy) endometrium |
| KUS | Patient K, endometriotic lesion isolated from |
| | uterosacral ligament |
| RH | Patient R, eutopic (healthy) endometrium |
| RRVS | Patient R, endometriotic lesion isolated from |
| | rectovaginal septum |
| RPF | Patient R, endometriotic lesion isolated from posterior formix |
| IH | Patient I, eutopic (healthy) endometrium |
| IV | Patient I, endometriotic lesion isolated from ovary |
| TH | Patient T, eutopic (healthy) endometrium |
| TPSWL | Patient T, endometriotic lesion isolated from pelvic side wall |
| PH | Patient P, eutopic (healthy) endometrium |
| PVL | Patient P, endometriotic lesion isolated from left ovary |
| PVR | Patient P, endometriotic lesion isolated from right ovary |
| LH | Patient L, eutopic (healthy) endometrium |
| LRV | Patient L, endometriotic lesion-1-isolated from |
| | rectovaginal region |
| LR | Patient L, endometriotic lesion isolated from the rectal region |
| NH | Patient N, eutopic (healthy) endometrium |
| NVR | Patient N, endometriotic lesion isolated from right ovary |
| OH | Patient O, eutopic (healthy) endometrium |
| OV | Patient O, endometriotic lesion isolated from ovary |

### Example 2

### Treatment of Endometrial Lesions in Baboons

A condition that closely resembles endometriosis in humans can be generated in baboons. In this model endometrial tissue is removed by biopsy during menstruation. This tissue is then introduced into the peritoneum of the same animal using a laporoscopic procedure. Endometrial lesions can begin to develop. If the procedure is repeated in the same animals at the time of subsequent menses then most if not all will develop advanced lesions. The basic techniques are described in more detail in D'Hooghe T. M. (1997) Fertil. Steril. 68 (4): 613-625 and Fazleabas A. T. (2002) Ann. NY Acad. Sci. 955: 308-317.

Endometriosis is thought to develop when fragments of endometrial tissue that are shed during the menstrual process, rather than passing out through the vagina, move retrogradely along the Fallopian tube and eventually enter the peritoneal cavity. The animal model used here mimics this process by directly introducing the endometrial tissue into the peritoneum.

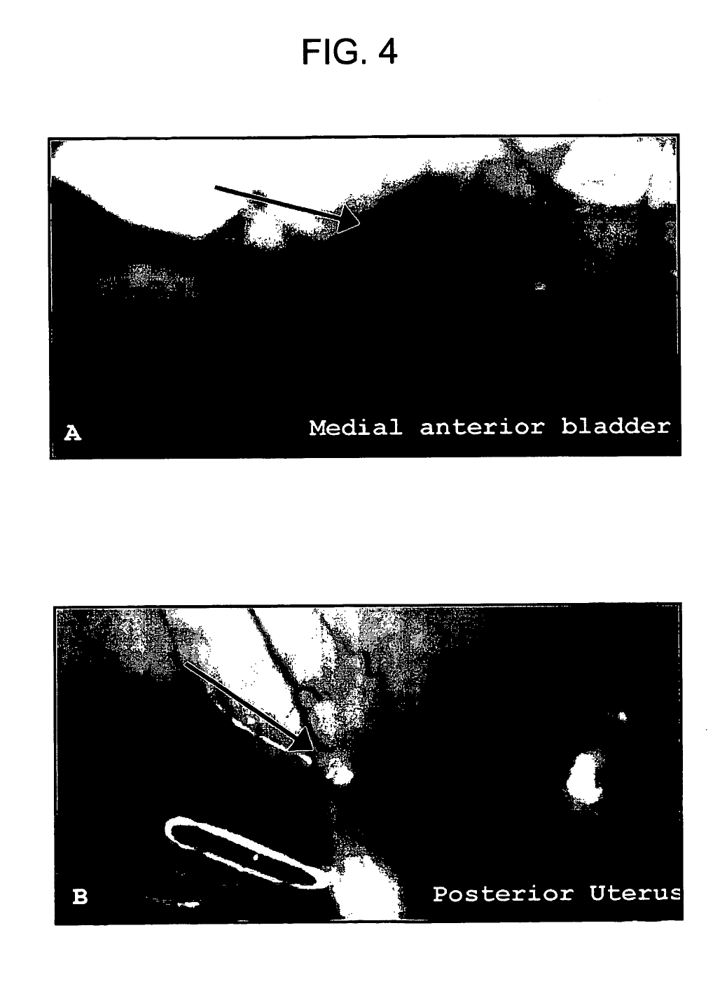

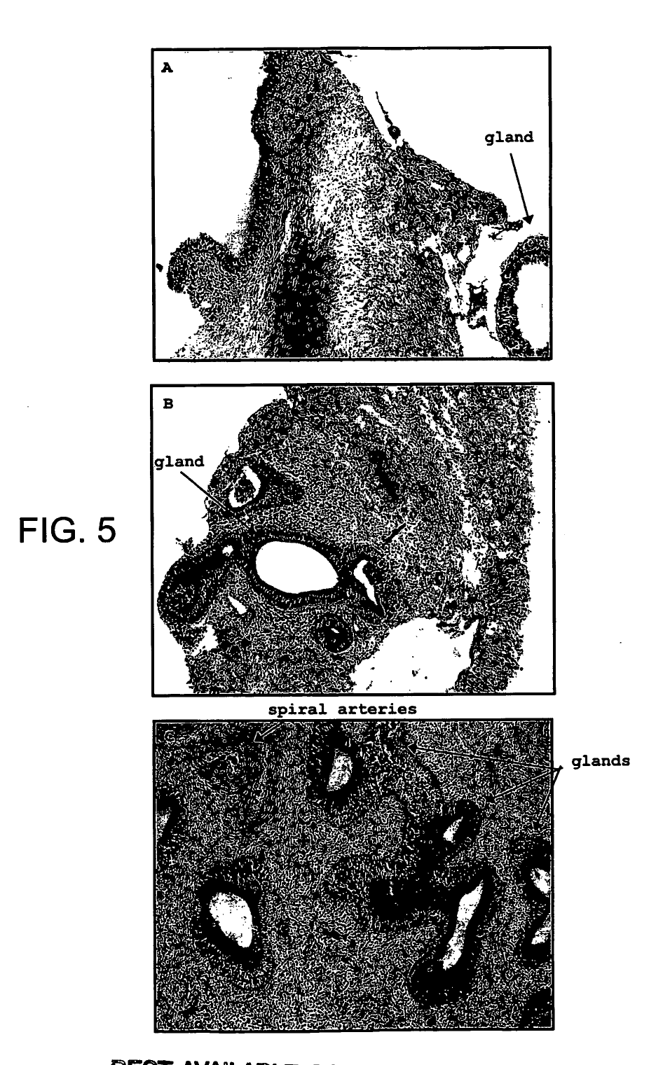

Endometriosis was induced in 3 baboons as follows. All three baboons were initially evaluated by laparoscopy for absence of lesions and adhesions. After the animals were confirmed to be clear of disease, endometrium was obtained during the first 2 days of menstruation by curettage from each animal and seeded back into the peritoneal cavity of the animal (first inoculation). At the next menses, a second evaluation laparoscopy was performed to document the presence of lesions and adhesions and the animals were inoculated for a second time with menstrual endometrium (second inoculation). One month after the second inoculation, all three animals were evaluated by laparoscopy for the presence of lesions and adhesions and disease was confirmed in all three animals. FIG. 4 shows in panel A a laparoscopic aspect of a particular lesion (lesion 5) in the medial anterior bladder of baboon PAN2615, whilst panel B shows a laparoscopic aspect of lesion 10 in the posterior uterus of the same baboon. FIG. 5 shows histological confirmation of the presence of endometrial glands in lesion 13 of baboon PAN2615 (panels A and B) and confirmation of the presence of glands in eutopic endometrium of this baboon (panel C). The presence of spiral arterioles is shown.

The timing of all procedures is shown below:

| | | | Aromatase |
|---|---|---|---|
| | First inoculation | Second inoculation | administration |
| Animal 1 | Day 1 | Day 36 | Day 99 |
| Animal 2 | Day 1 | Day 36 | Day 107 |
| Animal 3 | Day 1 | Day 35 | Day 108 |

The aromatase inhibitor used in this study was C14-labelled aminoglutethimide.

“D,L”-3-“4-Aminophenyl”-3-ethyl-2,6-piperidinedione[Phenyl-14C“U”] (aminoglutethamide) was dissolved in the minimal volume of dimethyl sulphoxide (DMSO). Specific activity was 57.8 mCi/mmol and radiochemical purity was >99% on HPLP. This solution was diluted into glycerol to provide the required concentrations and these resulting mixtures administered intra-vaginally to the anaesthetized animals. Systemic blood samples were taken at time intervals and at three hours, biopsies of endometrial lesions (and other tissue) were extirpated for radioactive counting.

Histology reveals significant variation between individual lesions and, not surprisingly, there are differences in the levels of radioactivity found in individual lesions. Some lesions will not be detected due to their anatomical location. In this study doses of 0.3, 0.6 and 0.9 mCi were administered to the individual animals and the numbers of obvious lesions identified were 1, 3 and 4 respectively.

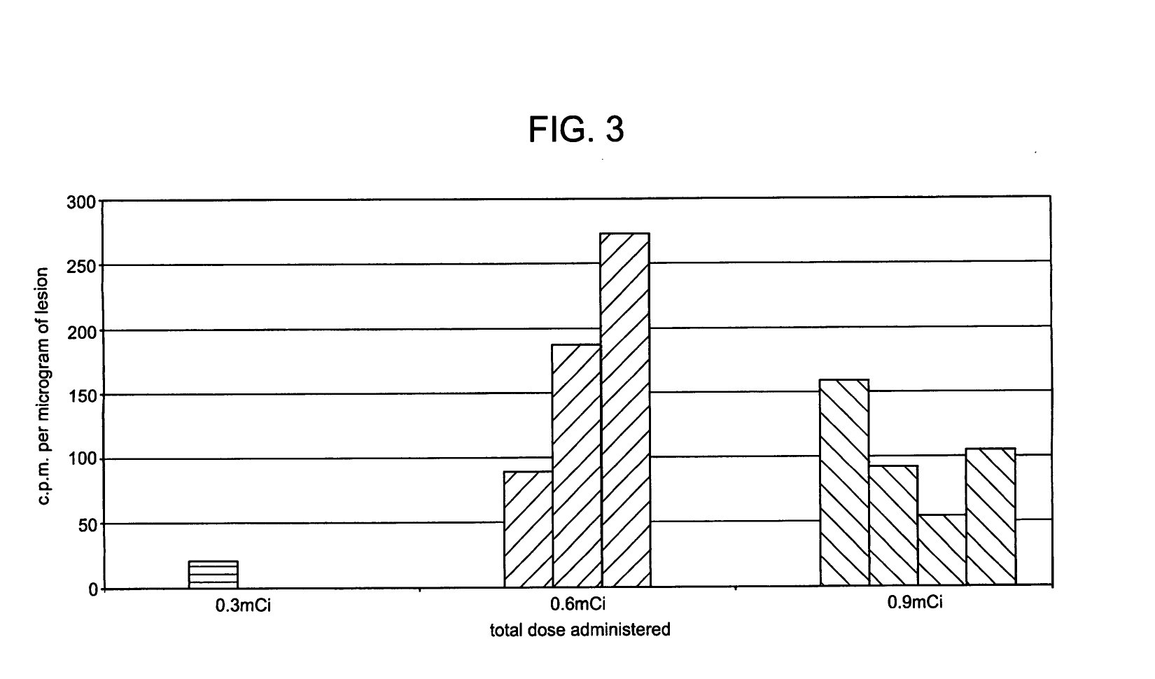

The results shown in FIG. 3 clearly show there has been transfer of aromatase inhibitor from vagina to endometrial lesions. Although blood levels were detectable and there was a dose-response relationship, levels never exceeded 2 c.p.m. per microgram confirming that the labelling of endometrial lesions has occurred via a direct drainage from vaginal mucosa rather than via systemic circulation. The fact that aromatase inhibitor was targetted to these lesions means that treatment at these sites was successfully effected.

1. A method of treating or preventing an oestrogen-dependent proliferative disorder of the uterus in a patient comprising administering to the patient an aromatase inhibitor intravaginally.

2. A method according to claim 1, wherein the oestrogen-dependent proliferative disorder of the uterus is endometriosis.

3. A method according to claim 1, wherein the oestrogen-dependent proliferative disorder of the uterus is uterine fibroids.

4. A method according to claim 1, wherein said aromatase inhibitor compounds is delivered intravaginally using an intra-vaginal delivery device.

5. A method according to claim 1, wherein said aromatase inhibitor compound is administered as part of a pharmaceutical formulation.

6. A method according to claim 5, wherein the pharmaceutical formulation contains a bioadhesive agent and/or absorption enhancer.

7. A method according to claim 1, wherein said aromatase inhibitor is a compound that inhibits the formation of an oestrogen from its precursor by an aromatase enzyme.

8. A method according to claim 7, wherein said aromatase inhibitor is selected from the group consisting of anastrozole; letrozole; exemestrane; vorozole; YM 511 (Yamanouchi Pharmaceutical); YM 553 (Yamanouchi Pharmaceutical); [(4-bromobenzyl)(4-cyanophenyl)amino]azoles and their azine analogs; 4-N-substituted amino-4H-1,2,4-triazole derivatives; 3-[N-(2-cholorbenzyl)amino]-6-(1H-imidazol-1-yl)pyridazine dihydrochloride (CAS 124070-28-3, MFF-279); aminoglutethimide; 4-hydroxy-androstenedione; 4-hydroxy-4-androstene-3,17-dione; 4-acetoxy-4-androstene-3,17-dione; fadrozole hydrochloride (CGS 16949A) (Bonzol; Mitsubishi-Tokyo Pharmaceuticals Inc); formestane; 1-methylandrosta-1,4-diene-3,17-dione; 1-methylandrosta-1,4-diene-3, 17-dione 17a-oxa-D-homooandrosta-1,4-diene-3,17-dione; androsta-4,6-diene-3,17-dione; androsta-4,6-dien-17.beta.-ol-3-one acetate; androsta-1,4,6-triene-3,17-dione; 4-androstene-19-chloro-3,17-dione; 4-androstene-3,6,17-trione; compounds described in “Endocrinology” (1973), vol. 92(3): 874; the 19-alkynylated steroids disclosed in German patent application 3,124,780; the 10-(1,2-propadienyl) steroids described in German patent application 3,124,719; the 19-thioandrostane derivatives described in EP-A-0,100,566; 4-androsten-4-ol-3,17-dione and its esters; the 1-methyl-15.alpha.-alkyl-androsta-1,4-diene-3,17-diones described in German patent application 3,539,244; the 10.beta.-alkynyl-4,9(11)-estradiene derivatives described in German patent application 3,644,358; 1,2.beta.-methylene-6-methylene-4-androstene-3,17-dione; or mixtures of any of these aromatase inhibitor compounds.

9. A method according to claim 1, wherein the amount of aromatase inhibitor that is administered in one dose is between 100 μg and 1_{g}, preferably between 100 μg and 10 mg.

10. A method according to claim 9, wherein the frequency of dosage is either daily, weekly, monthly, or quarterly (three monthly).

11. A pharmaceutical formulation comprising an aromatase inhibitor compound as recited in claim 1, for use in the treatment of an oestrogen-dependent proliferative disorder of the uterus by intravaginal administration.

12. (canceled)

13. An intra-vaginal device comprising an aromatase inhibitor compound in a therapeutically-effective amount.
